(19) United States
(12) Patent Application Publication
(10) Pub. No.: US 2010/0000303 A1
(43) Pub. Date: Jan. 7, 2010

(54) APPARATUS AND METHOD FOR DETERMINING THE PERCENTAGE OF CARBON EQUIVALENT, CARBON AND SILICON IN LIQUID FERROUS METAL

(76) Inventors: Anant Kashinath Kakatkar, Maharashtra State (IN); Satish Shashikant Kelkar, Maharashtra State (IN)

Correspondence Address: BROOKS KUSHMAN P.C., 1000 TOWN CENTER, TWENTY-SECOND FLOOR, SOUTHFIELD, MI, 48075, US

(21) Appl. No.: 12/443,608

(22) PCT Filed: Jul. 17, 2007
(86) PCT No.: PCT/IN2007/000295

§ 371 Date: Mar. 30, 2009

(30) Foreign Application Priority Data
Sep. 29, 2006 (IN) 1584/MUM/2006

(51) Int. Cl. G01N 33/20; G06F 19/00

(52) U.S. Cl. 73/61.76; 702/25

(57) ABSTRACT

The present invention relates to an apparatus and method for determining the percentage of Carbon Equivalent, Carbon and Silicon in liquid ferrous metal. The apparatus comprises of refractory cup structure having a cavity, thermocouple wire, quartz tube, base, tellurium, holder, compensating cable, electronic device. The method comprises steps of pouring of sample in a refractory cup, recording maximum temperature of the sample and allowing it to cool to solidification temperature, determination of liquidus temperature, this temperature being inversely proportional gives percentage of carbon equivalent using an algorithm, determination of solidus temperature using an algorithm, and the determination of percentage of carbon and silicon using electronic device based on an algorithm. The present invention relates to the detection of the composition of liquid ferrous metal in a much quicker time using a refractory cup.

### TECHNICAL FIELD

The present invention relates to an apparatus and method for determining the percentage of Carbon Equivalent, Carbon and Silicon in liquid ferrous metal. More particularly, the present invention relates to the detection of the composition of liquid ferrous metal in a much quicker time using refractory cups.

### BACKGROUND AND PRIOR ART

Existing thermal analysis for detection of percentage of carbon equivalent, carbon and silicon requires about 180 seconds and consumes about 200-325 grams of metal. Thermal analysis involves measurement and analysis of cooling pattern of the liquid metal, under controlled conditions. When liquid metal is poured in a cup, the maximum temperature as recorded by the cup is stored and further slow cooling is scanned. During cooling, when first nucleus solidifies, it gives away its kinetic energy, a thermal arrest is seen, which is metallurgically called as liquidus temperature (TL). This temperature being inversely proportional to the percentage of carbon equivalent (CE), helps in determining the % CE value. On further cooling, with the help of tellurium coating at the base of the cup, the sample is chilled and converted into white iron, instead of grey. This gives the solidification temperature (TS). The detection of TL and TS helps in detecting the percentage of Carbon and silicon.

The existing cups used are round or square in shape such that 225-325 grams of liquid metal can be accommodated for testing. Tellurium is pasted at the bottom of the cup or is coated all over the inner surface of the cup. The thermocouple wire used for measuring the temperature is a thick K type (CR-AL) wire of 22 SWG.

U.S. Pat. No. 3,404,570 discloses the method and apparatus for determining the concentration of a silicon in a sample of an electrically conductive material by measuring in a poured sample of the material cooling in a manner such that a temperature gradient exists across the sample, the temperature and a thermocouple force produced with the sample at each of two points in the sample spaced apart in the direction of the temperature gradient. The magnitude of the difference between the electromotive forces at a predetermined temperature difference between the points is representative of the concentration of the constituent.

However this method is complex and time consuming.

The present invention is fast and simple and takes only 50 to 180 seconds. The concept of finding composition in present invention is by thermal analysis as against conductivity measurement in prior art.

U.S. Pat. No. 3,546,921 discloses a method of producing an initial thermal arrest in the cooling curve of a molten sample of hypereutectic cast iron by addition of carbide stabilizers as Bi, B, Ce etc. are invented to get consistent discernible thermal arrest.

However this method applies only for determination of only Carbon Equivalent by determination of Liquidus temperature only. This invention does not determine % Carbon and % Silicon in the metal.

The present invention determines one more thermal arrest called solidus temperature. Moreover two additional elements viz. Carbon and silicon are determined as against only Carbon equivalent in prior art.

U.S. Pat. No. 4,059,996 sets forth an improvement over the other by disclosing a blob of material in contact with the bottom wall of a cavity. The blob of material includes a carbide formation promoting material and preferably mixed with a material for evolving hydrogen. The refractory material aids in preventing the carbide formation promoting material from being burned up quickly and mixing too quickly with the molten metal. The hydrogen thus evolved is used to generate turbulence in metal that help carbide forming material to reach to every corner of cup and thus achieve formation of carbides all over the cup.

The problem with this method is due to turbulence, the temperature drop is observed while filling the cup and cup filling is a skilled job. A stop and repeat pouring practice has to be followed to stop the boiling metal from coming out of the cup. All this is aimed at having uniform spreading of chilling agents throughout the cup.

In the present invention, turbulence is not created. The carbide forming material gets mixed without external force because of smaller volume of cup. This avoids generation of harmful hydrogen at such a high temperature of 1400° C. and splashing of metal from cup.

U.S. Pat. No. 4,515,485 also describe the improvement in U.S. Pat. No. 4,059,996 for mixing of chilling agents through out the cup by using evolved hydrogen in a better controlled fashion.

However it does not completely solve the problems associated with boiling and spilling of metal due to generation of hydrogen.

The present invention removes from root, the cause of creating turbulence by reducing the volume of cup that eliminates the requirement of generation of turbulence in the liquid metal.

U.S. Pat. No. 4,274,284 describe a method to improve response time of Cromel Alumel thermocouple that is used to measure temperature of the cup. High response time is very essential for accurate measurement of thermal arrests as described therein. The thermocouple is under constant thermal stress till analysis is complete.

This necessitates the use of thicker gauge causing response time and higher cost of wire and hence measurement. The gauge of the wire is more to withstand thermal stress as time requires for measurement is large.
However, the thermocouple remains exposed to liquid metal and thus the metal contaminates the thermocouple hampering accuracy.

In the present invention, volume of sample is reduced to 50 to 180 grams as against 200-325 grams as required by the prior art. Due to lower sampling time, the time for which the thermocouple has to undergo thermal stress reduces. A thinner thermocouple can be used due to lower exposure time. Secondly, a thinner wire has lesser lag and hence better response time. Hence objective of the prior art to reduce temperature lag is achieved automatically by reducing diameter of wire. The present invention allows using thinner thermocouples thereby reducing cost of sampling. The quartz tube (3) used eliminates contamination of carbonaceous material, which is another objective of prior art.

U.S. Pat. No. 6,739,750 provides a sampling vessel for thermal analysis of molten metal by reducing the time required with the help of probe type sampling vessel. The volume of the vessel is decided by the limitation in measurement accuracy of cooling rate. The cooling rate is required to be closer to (0 to −0.20 as mentioned in the FIG. 3 B). In the said process the conventional diameter of around 30 mm was reduced to around 20 mm and conventional depth of 50 mm was reduced to 36 mm or more.

The use of this technique involves the use of costlier probe type sample having a limitation of minimum depth of 36 mm of cavity.
With the present invention, by hardware and algorithm, cooling rate up to 30° C. is measured instead of 0.20° C., which eliminate the limitation of depth of 36 mm or more in prior art.

U.S. Pat. No. 5,720,553 describe the use of metallic inserts, instead of chilling agents, to act as a heat sink thereby promoting white solidification.

However, the cost of measurement is high and technique involves immersion type of sampling which is not preferred for measurement everywhere.
The present invention uses chilling agents and low volume of sample metal for promoting white solidification.

### DRAWBACKS OF PRIOR ART

1. The metal solidifies in the patches of grey and white iron which hampers accuracy of the testing to a great extent.
2. The pouring temperature of the metal is very high. It burns off some amount of tellurium thereby affecting quality of test.
3. The thermocouple is under constant thermal stress till analysis is complete.
4. The metal is held in the furnace for longer time which results in loss of electricity/power and deteriorates the quality of metal.
5. The thermal analysis requires more time.
6. The quantity of metal required for analysis is more.
7. The quantity of chilling agent required is more.

### SUMMARY OF THE PRESENT INVENTION

The main object of the present invention is to provide;

A) A method using refractory cup made from resin coated sand having capacity of 50 to 180 gm instead of prior art cup which needs quantity of 200-325 gm.
B) Hardware for determination of percentage of carbon, silicon and carbon equivalent.

Another object of the present invention is to increase the cooling rate by reducing the size of the resin coated cups. Lesser the volume, higher is the surface area to weight ratio and hence higher cooling rate is achieved.

Still further object of the present invention is to achieve balance in the pouring temperature such that temperature and time required is available for mixing of chilling agents and at the same time maximum cooling rate is achieved.

The purpose of the present invention is to reduce time needed for chilling material to mix at every corner of the cup in short time by reducing the distance of edges from centre of the cup by reducing the dimensions of the cup.

The aim of the present invention is to save time i.e. 50 to 80 seconds as against prior art, which needs 180 seconds and to save metal taken in the cavity for thermal analysis.

### ADVANTAGES OF THE PRESENT INVENTION

1. The metal solidifies into white iron as cooling rate is increased by reducing size of the cup and thereby reducing volume of the liquid metal which helps in accuracy of the testing.
2. The stress on the thermocouple last for a lesser time as time required for analysis is reduced due to faster cooling rate.
3. The time required for chilling material (tellurium) to mix at every corner of the cup is reduced as dimensions of the cup are changed.
4. The metal is held in furnace for shorter duration thereby saving electricity/power and helps in maintaining the quality of metal.
5. The quantity of metal required for analysis is less and thereby decrease in wastage of metal.
6. The quantity of chilling agents to convert gray iron to white iron is reduced.
7. The present invention thus provides convenient and rapid method for thermal analysis of a liquid ferrous metal.

### DESCRIPTION OF THE PRESENT INVENTION

According to the present invention for thermal analysis of a liquid ferrous metal, there is provided an apparatus and method for determining the concentration of a constituent in a liquid ferrous metal. More particularly present invention relates to a method and apparatus for determination of percentage of Carbon, Silicon and Carbon equivalent using electronic equipment.

### Apparatus:

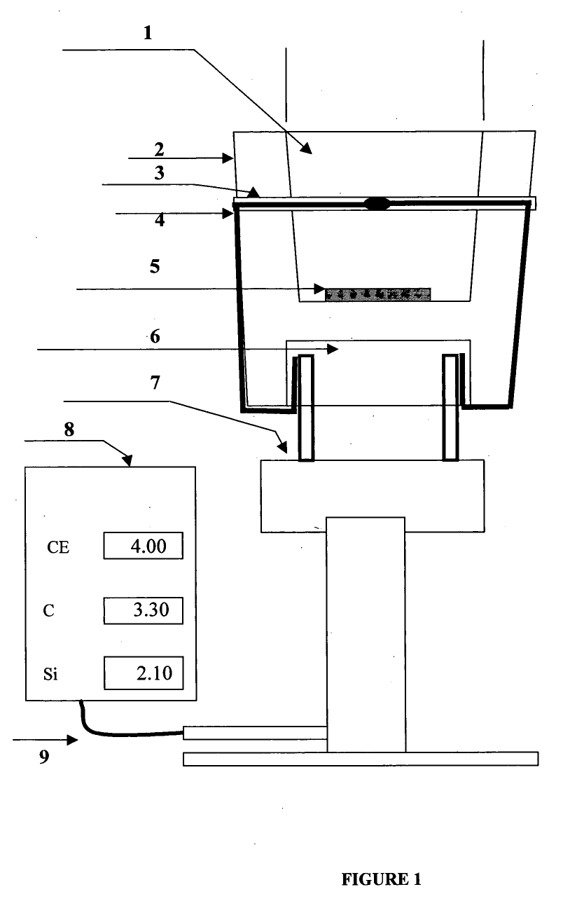

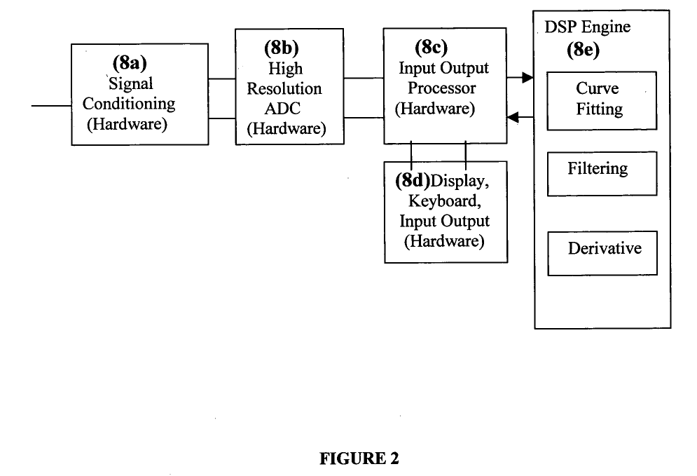

The apparatus of the present invention is illustrated in FIG. 1 of the accompanying drawing. FIG. 2 represents the block diagram of the electronic device.

The apparatus of the present invention comprises of well or mould or refractory cup structure (2), cavity (1), thermocouple wire (4), quartz tube (3), base (6), tellurium (5), holder (7), compensating cable (9) and electronic device (8).

The refractory cup structure or mould (2) is made from resin coated sand. The sand withstands high temperature of 1050 deg C. to 1400 deg C. as it is refractory in nature. The diameter and height of the cup structure (2) is around 20 to 40 mm and 10 to 25 mm respectively such that the weight of the metal in the cup is about 50 to 180 gm.

The K type (CR-AL) 22 to 24 swg thermocouple wire (4) is used for measuring the temperature. Quartz tube shell (3) is fitted horizontally in the cup structure (2) such that it covers CR-AL wire (4). The quartz tube (3) avoids contact of liquid ferrous metal and thermocouple wire (4) and eliminate possibility of contamination. The quartz tube (3) is sealed with refractory agents so that there is no leakage from hole of cup (2).

Chilling agents such as Bismuth, Boron, Cerium, Lead, Magnesium and Tellurium (5) are mixed with refractory binders is pasted at the bottom of the cup as chilling agent. The quantity of chilling agents used is 0.20 to 0.50 gm. (0.2 to 0.6% by weight).

The refractory cup (2) has a suitable base (6) so as to fit it to the holder (7). This holder then carries signal to the electronic device (8) via compensating cable (9) for further analysis of percentage of Carbon, Silicon and Carbon equivalent.

An electronic device (8) capable of sensing thermal arrest points at high cooling rates is connected to the holder (7) through a compensating cable (9). This electronic device (8) finds the Liquidus and solidus temperature as per algorithm, store, convert and display corresponding values of % CE, % Carbon and % Si on the display.

Electronic device (8) comprises of signal conditioning hardware (8a), analog to digital converter (8b), input output processor (8c), display (8d), and digital signal processor (8e).

### Method:

The liquid ferrous metal sample is poured in a cavity (1) of cup (2). The maximum temperature as recorded by the cup (2) is stored in the electronic device (8) and further cooling is scanned. The heat liberated when austenite starts to precipitate produces an isothermal arrest on the cooling curve. During solidification in the cup, latent heat is given out. Due to the effect of natural cooling and liberation of latent heat, a thermal equilibrium is reached and a thermal arrest is obtained. This temperature is called as Liquidus temperature (TL). The arrest found, according to this invention is relatively weak due to faster cooling rate. This weak arrest is due to lower weight of sample and hence lower latent heat available to arrest temperature. The liquidus temperature being inversely proportional to the % carbon equivalent (CE), determine the % CE value empirically.

The sample gets chilled with the help of chilling agents (5) coating at the bottom of the cup (2) from inside the cavity and the sample converted into white iron. When all the liquid metal solidifies one more thermal arrest is obtained. This temperature is called Solidus temperature (TS). The time required for analysis to complete is about 50 to 80 seconds.

It is a property of any substance to have a fixed freezing point. But Cast iron, S.G Iron, malleable iron that is under consideration of present invention is exception to it. Generally iron of the consideration in present invention solidifies showing grey structure when fractured. In such case, iron with same composition solidifies, at different temperature depending upon nucleation. More the nucleation, higher is the freezing point. But when it is allowed to cool fast, it solidifies giving white fracture. It is called metastable solidification. Iron with same composition solidifies at a unique temperature if it is allowed to solidify at metastable solidification temperature.

The universal Iron Carbon diagram/Iron Carbon Silicon diagram shows different solidification compositions for different values of liquidus and solidus temperature for white solidification.

The present invention makes use of metastable solidification. The metal is forced to cool fast using chilling agents. This causes metastable solidification to occur. The instrument senses solidification temperature of the iron. A table of different values of solidification temperatures verses their corresponding composition is fed in the instrument. The algorithm searches for stored liquidus and solidus temperature values and locates corresponding values of % Carbon Equivalent, % Carbon.

The value of % Si is calculated by using formula

% Carbon Equivalent=% Carbon+(⅓)*% Silicon.

Smaller quantity of sample considered in the present invention cool faster than conventional sample quantity. Hence present invention uses the instrument, which can process higher cooling rates.

The faster cooling rates are measured due to the lower quantity of the sample under test. The hardware and the algorithm used in the present invention can handle cooling rates of 0 to 3° C./sec while finding liquidus and solidus temperatures.

The method using hardware and algorithm for the complete process is described in details herein.
1. When liquid metal is poured in the cup (2), the thermocouple inside the cup (2) gets heated up and generates signal in millivolts.
2. Signal conditioning (8a) of the millivolts is carried out using various components like filters and capacitors.
3. Analog signal is converted to digital signal using high resolution analog to digital converter (8b) so that input output processor (8c) can process it.
4. Store all the points of cooling process in an array using input output processor (8c).
5. A curve is generated using digital signal processor (8e) engine. Interpolation of the intermediate points is done to smoothen the curve.
6. The instantaneous cooling rate i.e 1^{st} derivative at each point of the smoothened cooling curve is done by digital signal processor (8e) engine and the cooling rate values are stored in another array.
7. A filter is applied by the digital signal processor (8e) engine to fit a smooth curve for 1^{st} derivative graph by interpolation.
8. 2^{nd} derivative at each point of the smoothened 1^{st} derivative curve is found and the 2^{nd} derivative values are stored in another array.
9. A filter is applied by the digital signal processor (8e) engine to fit a smooth curve for 2^{nd} derivative graph by interpolation and the values obtained are stored in another array.
10. 3^{rd} derivative at each point of the smoothened 2^{nd} derivative curve is found with the help of digital signal processor (8e) engine and the 3^{rd} derivative values are stored in another array.
11. Maxima, minima and zero crossover points of cooling rate, 1st and 2^{nd} derivative curves are found by using digital signal processor (8e) engine.
12. liquidus temperature and solidus temperature are detected using above mentioned points.

Liquidus and solidus point detection: When iron containing Carbon and silicon solidifies, it does so over the range of temperature instead of solidifying at a particular freezing point. When material is poured in the cup (2), the electronic device (8) senses the maximum temperature. When material is allowed to cool, initially it starts cooling at maximum cooling rate. When the temperature reaches the solidification temperature, few molecules start to solidify to precipitate austenite and thus give out latent heat of solidification. The resultant of natural cooling of material and evaluation of latent heat reduce the cooling rate of solidifying metal. Depending upon the quantity of latent heat available with the solidifying metal, the cooling rate start falling down, reach to a minimum level and start raising again. The temperature of lowest achieved cooling rate is the liquidus temperature. Since the reading are stored as time V/s temperature, the first derivative of these points is cooling rate and 2^{nd} derivative is rate of change of cooling rate. Therefore when the 2^{nd} derivative passes through zero the minima on the cooling rate curve is obtained. Corresponding temperature is the liquidus temperature.

With the same principle the solidus temperature is found. When material cool further, it reaches a temperature where the material is completely solid. It again gives out heat and the cooling rate drop again. This change in cooling rate is sensed and latched as solidus temperature.

Using algorithm, cooling rate from 0 to 3 deg. C./Sec. can be measured, handled, analyzed used by input output processor (8c) of electronic device (8) for detecting liquidus and solidus temperatures.
13. The empirical table of temperature verses corresponding % carbon equivalent and % carbon are stored in input output processor using iron carbon diagram. Input output processor (8c) find corresponding value of carbon equivalent by using liquidus temperature and display its value on the electronic device (8).
14. Input output processor (8c) is used to find % Silicon using following formula.

% Carbon Equivalent=% Carbon+(⅓)*% Silicon.
15. Input output processor (8c) send values of % carbon & % silicon, liquidus and solidus temperature and display values on the display (8d) of electronic device (8).

The important processing in this hardware and algorithm essentially lies in step 5, where a filter is applied and a smooth curve fit is generated. This algorithm ensures more precise values when working with higher cooling rate. The algorithm is capable of detecting liquidus temperature and solidus temperature up to cooling rate of 3 deg./sec while finding liquidus and solidus temperatures.

Detailed descriptions of the preferred embodiment are provided herein; however, it is to be understood that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or matter.

The embodiments of the invention as described above and the method disclosed herein will suggest further modification and alterations to those skilled in the art. Such further modifications and alterations may be made without departing from the spirit and scope of the invention; which is defined by the scope of the following claims.

1. An apparatus for determining the percentage of Carbon Equivalent, Carbon and Silicon in liquid ferrous metal comprising:
a refractory cup structure mould or well having a cavity; a thermocouple wire; a quartz tube; a base; chilling agents, holder, compensating cable, and an electronic device;
wherein the refractory cup structure is made up of sand coated with resin; the diameter and height of the said cup structure is around 20 to 40 mm and 10 to 25 mm respectively; the cavity is such that weight of the liquid metal or sample is about 50 to 180 gm; the K type (CR-AL) 22 to 24 SWG thermocouple wire is used for measuring the temperature; quartz tube shell is fitted horizontally in the said cup structure such that it covers said CR-AL wire; the said quartz tube avoids contact of said liquid ferrous metal and said thermocouple wire and eliminates possibility of contamination; the said quartz tube is sealed with refractory agents so that there is no leakage from hole of the said cup structure; Chilling agents 0.20 to 0.50 gm mixed with refractory binders is pasted at the bottom of the said cup; the said cup has a suitable base so as to fit it to the holder; this said holder then carry signal to the electronic device via compensating cable for further analysis of percentage of Carbon equivalent, Carbon and Silicon.

2. An apparatus as claimed in claim 1 where the said electronic device comprises:
a signal conditioning hardware;
an analog to digital converter;
an input output processor;
a display; and
a digital signal processor.

3. A method using apparatus as claimed in claim 1 for determining the percentage of Carbon equivalent, Carbon and Silicon in liquid ferrous metal comprising steps of;
a. when liquid metal is poured in the said cup, the thermocouple inside the said cup gets heated up and generates signal in millivolts;
b. signal conditioning of the millivolts is carried out using various components like filters and capacitors;
c. analog signal is converted to digital signal using high resolution analog to digital converter so that input output processor can process it;
d. store all the points of cooling process in an array using input output processor;
e. a curve is generated using digital signal processor engine; Interpolation of the intermediate points is done to smoothen the curve;
f. the instantaneous cooling rate i.e 1^{st} derivative at each point of the smoothened cooling curve is done by said digital signal processor engine and the cooling rate values are stored in another array;
g. a filter is applied by the said digital signal processor engine to fit a smooth curve for 1^{st} derivative graph by interpolation;
h. 2^{nd} derivative at each point of the smoothened 1^{st} derivative curve is found and the 2^{nd} derivative values are stored in another array;
i. a filter is applied by the said digital signal processor engine to fit a smooth curve for 2^{nd} derivative graph by interpolation and the values obtained are stored in another array;
j. 3^{rd} derivative at each point of the smoothened 2^{nd} derivative curve is found with the help of said digital signal processor engine and the 3^{rd} derivative values are stored in another array;
k. maxima, minima and zero crossover points of cooling rate, 1^{st} and 2^{nd} derivative curves are found by using said digital signal processor engine;
l. liquidus temperature and solidus temperature are detected using above mentioned points;
liquidus and solidus point detection: When iron containing Carbon and silicon solidify, it does so over the range of temperature instead of solidifying at a particular freezing point; when material is poured in the said cup, the said electronic device senses the maximum temperature; when material is allowed to cool, initially it starts cooling at maximum cooling rate; when the temperature reaches the solidification temperature, few molecules start to solidify to precipitate austenite and thus give out latent heat of solidification; the resultant of natural cooling of material and evaluation of latent heat reduce the cooling rate of solidifying metal; depending upon the quantity of latent heat available with the solidifying metal, the cooling rate start falling down, reach to a minimum level and start raising again; the temperature of lowest achieved cooling rate is the liquidus temperature; since the reading are stored as time V/s temperature, the first derivative of these points is cooling rate and 2^{nd} derivative is rate of change of cooling rate; therefore when the 2^{nd} derivative passes through zero the minima on the cooling rate curve is obtained; corresponding temperature is the liquidus temperature;
with the same principle the solidus temperature is found; when material cools further, it reaches a temperature where the material is completely solid; it again gives out heat and the cooling rate drop again; this change in cooling rate is sensed and latched as solidus temperature;
using algorithm, cooling rate from 0 to 3 deg. C./Sec. can be measured, handled, analyzed used by said input output processor of said electronic device for detecting liquidus and solidus temperatures;
m. the empirical table of temperature verses corresponding % carbon equivalent and % carbon are stored in said input output processor using iron carbon diagram; the said input output processor find corresponding value of carbon equivalent by using liquidus temperature and display its value on the said electronic device;
n. the said input output processors used to find % Silicon using following formula; % Carbon Equivalent=% Carbon+(⅓)*% Silicon; and
o. the said input output processor send values of % carbon & % silicon, liquidus and solidus temperature and display values on the display of said electronic device.

4. An apparatus as claimed in claim 1 where the cup structure is polygonal.

5. An apparatus as claimed in claim 1 wherein instead of tellurium other chilling agents e.g. Bismuth, Boron, Cerium, Lead, and Magnesium or alike can be used as an alternative.

6. An apparatus as claimed in claim 1 wherein instead of (CR-AL) 22 or 24 SWG thermocouple other thermocouple capable of measurement in the range of 1050 to 1400 deg C.—can be used as an alternative.

7. An apparatus as claimed in claim 1 wherein the time of measuring percentage of Carbon Equivalent, Carbon and Silicon is from 50 to 180 seconds.

8. (canceled)
